SEQUENCE LISTING (24 sequences)

SEQ ID NO: 1 - protein, 120 aa, Artificial Sequence
  Synthetic Construct
QDQLVESGGGVVQPGRSLRLSCAASGFTFSSYGMHWVRQAPGEGLEWVAVIWYDGSNKYYADSVKGRFTISRDNSKNTLYLQMNSLRAEDTAVYYCARGDYEVDYGMDVWGQGTTVTVAS

SEQ ID NO: 2 - protein, 107 aa, Artificial Sequence
  Synthetic Construct
AIQLTQSPSSLSASVGDRVTITCRASQGISNALAWYQQKPGKAPKLLIYDASSLESGVPSRFSGSGSGTDFTLTISSLQPEDFATYYCQQFNSYPWTFGQGTKVEIK

SEQ ID NO: 3 - protein, 450 aa, Artificial Sequence
  Synthetic Construct
QDQLVESGGGVVQPGRSLRLSCAASGFTFSSYGMHWVRQAPGEGLEWVAVIWYDGSNKYYADSVKGRFTISRDNSKNTLYLQMNSLRAEDTAVYYCARGDYEVDYGMDVWGQGTTVTVASASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 214 aa, Artificial Sequence
  Synthetic Construct
AIQLTQSPSSLSASVGDRVTITCRASQGISNALAWYQQKPGKAPKLLIYDASSLESGVPSRFSGSGSGTDFTLTISSLQPEDFATYYCQQFNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 123 aa, Artificial Sequence
  Synthetic Construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARSPDYSPYYYYGMDVWGQGTTVTVSS

SEQ ID NO: 6 - protein, 111 aa, Artificial Sequence
  Synthetic Construct
QSVLTQPPSASGTPGQRVTISCSGSSSNIGSNTVNWYQQLPGTAPKLLIYGNSNRPSGVPDRFSGSKSGTSASLAISGLQSEDEADYYCQSYDSSLSGSVFGGGIKLTVLG

SEQ ID NO: 7 - protein, 453 aa, Artificial Sequence
  Synthetic Construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARSPDYSPYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 216 aa, Artificial Sequence
  Synthetic Construct
QSVLTQPPSASGTPGQRVTISCSGSSSNIGSNTVNWYQQLPGTAPKLLIYGNSNRPSGVPDRFSGSKSGTSASLAISGLQSEDEADYYCQSYDSSLSGSVFGGGIKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECS

SEQ ID NO: 9 - protein, 119 aa, Artificial Sequence
  Synthetic construct
DRQMVESGGGLVRPGRSLKLSCAASGFTFSNYYMAWVRLAPSKGLEWVASISPSGDNTYYPDSVQGRFTISRDNAETTLYLQVDSLRSEDTATYYCATRHYGYSFFDYWGQGVLVTVSS

SEQ ID NO: 10 - protein, 106 aa, Artificial Sequence
  Synthetic construct
DTVLTQSPALAVSPGERVTISCRASESVSTLIHWYQQKPGQQPKLLIFLASHLESGVPARFSGSGSGTDFTFTIDPVEADDTATYYCQQSWNDPPTFGGGTKLELK

SEQ ID NO: 11 - protein, 443 aa, Artificial Sequence
  Synthetic construct
QVQLQQSGADLAKPGSSVKISCKASGYNFNSYYINWIKQTTGQGLEYIGYINTVSGTTKYSEKFKGKATLTVDKSSSTAFMQLSSLTPDDSAVYYCARGTIVLDDYWGQGVKVTVSSAETTAPSVYPLAPGTALKSNSMVTLGCLVKGYFPEPVTVTWNSGALSSGVHTFPAVLQSGLYTLTSSVTVPSSTWPSQTVTCNVAHPASSTKVDKKIVPRNCGGDCKPCICTGSEVSSVFIFPSKPKDVLTITLTPKVTCVVVDISQDDPEVHFSWFVDDVEVHTAQTRPPEEQFNSTFRSVSELPILHQDWLNGRTFRCKVTSAAFPSPIEKTISKPEGRTQVPHVYTMSPTKEEMTQNEVSITCMVKGFYPPDIYVEWQMNGQPQENYKNTPPTMDTDGSYFLYSKLNVKKEKWQQGNTFTCSVLHEGLHNHHTEKSLSHSPGK

SEQ ID NO: 12 - protein, 220 aa, Artificial Sequence
  Synthetic construct
DIVMTQTPSSQAVSAGEKVTMSCKSSQSLLYNEKKKNYLAWYQQKPGQSPKLLIYWASTRESGVPDRFLGSGSGTDFTLTINSVQAEDLAVYYCQQSYDFPRTFGGGTKLELKRADAAPTVSIFPPSTEQLATGGASVVCLMNNFYPRDISVKWKIDGTERRDGVLDSVTDQDSKDSTYSMSSTLSLTKADYESHNLYTCEVVHKTSSSPVVKSFNRNEC

SEQ ID NO: 13 - protein, 5 aa, Artificial Sequence
  Synthetic construct
SYGMH

SEQ ID NO: 14 - protein, 17 aa, Artificial Sequence
  Synthetic construct
VIWYDGSNKYYADSVKG

SEQ ID NO: 15 - protein, 11 aa, Artificial Sequence
  Synthetic construct
GDYEVDYGMDV

SEQ ID NO: 16 - protein, 11 aa, Artificial Sequence
  Synthetic construct
RASQGISNALA

SEQ ID NO: 17 - protein, 7 aa, Artificial Sequence
  Synthetic construct
DASSLES

SEQ ID NO: 18 - protein, 9 aa, Artificial Sequence
  Synthetic construct
QQFNSYPWT

SEQ ID NO: 19 - protein, 13 aa, Artificial Sequence
  Synthetic construct
KASGGTFSSYAIS

SEQ ID NO: 20 - protein, 17 aa, Artificial Sequence
  Synthetic construct
GIIPIFGTANYAQKFQG

SEQ ID NO: 21 - protein, 16 aa, Artificial Sequence
  Synthetic construct
ARSPDYSPYYYYGMDV

SEQ ID NO: 22 - protein, 13 aa, Artificial Sequence
  Synthetic construct
SGSSSNIGSNTVN

SEQ ID NO: 23 - protein, 8 aa, Artificial Sequence
  Synthetic construct
YGNSNRPS

SEQ ID NO: 24 - protein, 11 aa, Artificial Sequence
  Synthetic construct
QSYDSSLSGSV

(19) United States
(12) Patent Application Publication
(10) Pub. No.: US 2018/0291107 A1
(43) Pub. Date: Oct. 11, 2018

(54) COMBINATION THERAPY FOR CANCER

(71) Applicant: Eli Lilly and Company, Indianapolis, IN (US)

(72) Inventor: David Arlen SCHAER, Mamaroneck, NY (US)

(21) Appl. No.: 15/757,146

(22) PCT Filed: Nov. 17, 2016
(86) PCT No.: PCT/US2016/062401

§ 371 Date: Mar. 2, 2018

Related U.S. Application Data
(60) Provisional application No. 62/259,379, filed on Nov. 24, 2015.

(51) Int. Cl. C07K 16/28; A61P 35/00

(52) U.S. Cl. CPC C07K 16/2866; A61K 2039/507; C07K 16/2827; A61P 35/00

(57) ABSTRACT

The present invention relates to the field of medicine. More particularly, the present disclosure relates to combinations of an antibody that binds colony-stimulating factor 1 receptor (CSF-IR) and an antibody that binds human programmed cell death I ligand 1 (PD-L1) and to methods of using the combination to treat cancer, and in particular solid tumors with macrophage infiltration.

The present invention relates to the field of medicine. More particularly, the present invention relates to combinations of an antibody that binds colony-stimulating factor 1 receptor (CSF-1R) and an antibody that binds human programmed cell death 1 ligand 1 (PD-L1), and to methods of using the combination to treat cancer, and in particular solid tumors and tumors with macrophage infiltration.

Macrophages are among the immune cells that infiltrate solid tumors. In many cancers, higher levels of macrophage infiltration have been associated with poorer prognosis; head and neck, skin, melanoma, mesothelioma, breast, ovarian, uterine, cervical, bladder, kidney, pancreatic, liver, thyroid and brain are among the primary cancers that have shown to have a clinical outcome adversely affected by the presence of macrophages. Ruffell & Coussens, Cancer Cell (2015) 27 (4):462.

Tumor-associated macrophages (TAM) are abundant in tumors, and promote growth, angiogenesis, and metastasis through secretion of proangiogenic factors and remodeling of the tumor stroma. In addition, TAMs are known to cause suppression of anticancer immune responses through direct inhibition of anti-tumor T cells by production of reactive oxygen species and suppressive cytokines. Id. Therefore, TAMs have been identified as a potential target for cancer therapeutics.

CSF-1R, also known as M-CSFR or CD-115, is a tyrosine kinase receptor expressed selectively on macrophage and granulocyte cell lineages in normal individuals and on tumor cells in cancer. Upon ligand binding, CSF-1R dimerizes, leading to trans-phosphorylation of the receptor and phosphorylation and activation of downstream signaling molecules such as MAPK and Akt. Phosphorylation of CSF-1R results in: (1) the proliferation and differentiation of macrophages from hematopoietic progenitor stem cells, (2) survival and migration of macrophages to various organs and tissues in the body, particularly the tumor stroma, and (3) maintenance of the immune suppressive phenotype of TAM and other CSF-1R expressing cells of the myeloid lineage. Given the critical role of CSF-1R in the regulation and survival of TAMs and other myeloid cells, CSF-1R inhibition has been identified as a potential cancer target.

Programmed cell death 1 (PD-1) and PD-L1 are part of an immune checkpoint pathway used normally in maintenance of self-tolerance and control of T cell activation, but cancer cells can use the pathway to suppress the anti-tumor response and prevent their destruction. Clinical research has found that targeting the PD-1/PD-L1 axis with antagonist antibodies to either protein can cause tumor regression in patients and prolong survival. However, many patients still do not receive a benefit. CSF-1R expression may be one factor that impacts response. CSF-1R expressing cells of the myeloid linage, including TAM and myeloid derived suppressor cells (MDSCs) have been implicated in potentially contributing to the immune suppression that prevent the effectiveness of PD-1/PD-L1 targeted therapies.

In a mouse model of pancreatic ductal adenocarcinoma, inhibition of CSF-1R was reported to increase PD-L1 levels, and treatment with a small molecule CSF-1R inhibitor and a PD-1 antagonist improved efficacy over CSF-1R inhibition alone (Zhu et al., Cancer Res (2014) 74(18):5057). However, the nonspecific potential of a small molecule CSF-1R inhibitor provides nominal mechanistic evidence that the interaction between CSF-1R and PD-1 drove the improved efficacy. In fact, in a mouse CT26 colon carcinoma model, a PD-L1 antibody alone was compared to CSF-1R antibody and a PD-L1 antibody (US 2015/0073129) and only a slight improvement was shown (PD-L1 antibody: 83% tumor growth inhibition, 0.27 tumor to control ratio, and 32 days to progression >700 mm³, PD-L1 antibody and CSF-1R antibody: 83% tumor growth inhibition, 0.28 tumor to control ratio, and 37 days to progression >700 mm³). For a CT26 colon carcinoma model (US 2015/0073129), there was no reported difference in median time to progression for PD-L1 antibody alone or PD-L1 antibody plus CSF-1R antibody.

Unfortunately, despite more therapeutic options for solid tumors using immunotherapy, treatment options for indications where macrophages play a major role in immune suppression still remain elusive. There exists a need for more and different therapies that can effectively eliminate the influence of TAM in the tumor microenvironment and relieve the suppression they exert. TAMs and MDSC also appear to be an adaptive mechanism of suppression that is activated during immunotherapy and limits its usefulness. Targeting these cells could increase the number of patients that respond to immunotherapy and prevent recurrence. Targeting CSF-1R on TAMs and MDSCs could relieve immune suppression of anti-tumor T cells, reducing their exhaustion in the tumor and enhancing their activity.

There remains a need to provide alternative combinations of CSF-1R and PD-L1 antibodies. In particular, there remains a need to provide combinations of CSF-1R and PD-L1 antibodies that may be able to affect a durable complete regression of tumor burden which may prevent recurrence or result in control of tumor burden producing clinical benefit. There remains a need to provide combinations of CSF-1R and PD-L1 antibodies where lower dosing of the CSF-1R antibody can be achieved, thereby potentially reducing toxicity. There remains a need to provide a combination of therapies that can effectively deplete macrophages and relieve inhibition, and prevent T cell exhaustion in the tumors.

Accordingly, in some embodiments the present invention provides a method of treating cancer, comprising administering in simultaneous, separate, or sequential combination to a patient in need thereof, effective amounts of a first antibody that binds to human CSF-1R and a second antibody that binds to human PD-L1, wherein: the first antibody comprises a light chain (LC) and a heavy chain (HC), wherein the light chain comprises a light chain variable region (LCVR) and the heavy chain comprises a heavy chain variable region (HCVR), and wherein the LCVR comprises light chain complementarity determining regions LCDR1, LCDR2, and LCDR3 consisting of the amino acid sequences RASQGISNALA (SEQ ID NO: 16), DASSLES (SEQ ID NO: 17), and QQFNSYPWT (SEQ ID NO: 18), respectively, and wherein the HCVR comprises heavy chain complementarity determining regions HCDR1, HCDR2, and HCDR3 consisting of the amino acid sequences SYGMH (SEQ ID NO: 13), VIWYDGSNKYYADSVKG (SEQ ID NO: 14), and GDYEVDYGMDV (SEQ ID NO: 15), respectively; and the second antibody comprises a LC and a HC, wherein the LC comprises a LCVR and the HC comprises a HCVR, and wherein the LCVR comprises light chain complementarity determining regions LCDR1, LCDR2, and LCDR3 consisting of the amino acid sequences SGSSSNIGSNTVN (SEQ ID NO: 22), YGNSNRPS (SEQ ID NO: 23), and QSYDSSLSGSV (SEQ ID NO: 24), respectively, and wherein the HCVR comprises heavy chain complementarity determining regions HCDR1, HCDR2, and HCDR3 consisting of the amino acid sequences KASGGTFSSYAIS (SEQ ID NO: 19), GIIPIFGTANYAQKFQG (SEQ ID NO: 20), and ARSPDYSPYYYYGMDV (SEQ ID NO: 21), respectively.

In some embodiments the present invention provides a method of treating cancer, comprising administering in simultaneous, separate, or sequential combination to a patient in need thereof; effective amounts of a first antibody and a second antibody, wherein: the first antibody comprises a LC and a HC, wherein the LC comprises a LCVR and the HC comprises a HCVR, wherein the LCVR has the amino acid sequence given in SEQ ID NO: 2, and the HCVR has the amino acid sequence given in SEQ ID NO: 1; and the second antibody comprises a LC and a HC, wherein the LC comprises a LCVR and the HC comprises HCVR, wherein the LCVR has the amino acid sequence given in SEQ ID NO: 6, and the HCVR has the amino acid sequence given in SEQ ID NO: 5.

In a further embodiment, the present invention provides a method of treating cancer wherein the LC of the first antibody has the amino acid sequence given in SEQ ID NO: 4, and the HC of the first antibody has the amino acid sequence given in SEQ ID NO: 3. In a further embodiment, the present invention provides a method of treating cancer wherein the LC of the second antibody has the amino acid sequence given in SEQ ID NO: 8, and the HC of the second antibody has the amino acid sequence given in SEQ ID NO: 7.

In a further embodiment, the present invention provides a method of treating cancer wherein: the first antibody comprises two light chains and two heavy chains, wherein each light chain has the amino acid sequence given in SEQ ID NO: 4, and each heavy chain has the amino acid sequence given in SEQ ID NO: 3; and the second antibody comprises two light chains and two heavy chains, wherein each light chain has the amino acid sequence given in SEQ ID NO: 8, and each heavy chain has the amino acid sequence given in SEQ ID NO: 7.

In some embodiments the present invention provides a method of treating cancer, comprising administering in simultaneous, separate, or sequential combination to a patient in need thereof, effective amounts of a first antibody that binds to human CSF-1R and a second antibody that binds to human PD-L1, wherein: the first antibody comprises a LC and a HC, wherein the LC comprises a LCVR and the HC comprises a HCVR, wherein the LCVR has the amino acid sequence given in SEQ ID NO: 2, and the HCVR has the amino acid sequence given in SEQ ID NO: 1; and the second antibody comprises a LC and a HC, wherein the LC comprises a LCVR and the HC comprises a HCVR, wherein the LCVR has the amino acid sequence given in SEQ ID NO: 6, and the HCVR has the amino acid sequence given in SEQ ID NO: 5.

In a further embodiment, the present invention provides a method of treating cancer wherein: the first antibody that binds to human CSF-1R comprises a LC and a HC, wherein the LC has the amino acid sequence given in SEQ ID NO: 4, and the HC has the amino acid sequence given in SEQ ID NO: 3; and the second antibody that binds to human PD-L1 comprises a LC and a HC, wherein the LC has the amino acid sequence given in SEQ ID NO: 8, and the HC has the amino acid sequence given in SEQ ID NO: 7.

In a further embodiment, the present invention provides a method of treating cancer wherein: the first antibody that binds to human CSF-1R comprises two light chains and two heavy chains, wherein each light chain has the amino acid sequence given in SEQ ID NO: 4, and each heavy chain has the amino acid sequence given in SEQ ID NO: 3; and the second antibody that binds to human PD-L1 comprises two light chains and two heavy chains, wherein each light chain has the amino acid sequence given in SEQ ID NO: 8, and each heavy chain has the amino acid sequence given in SEQ ID NO: 7.

In a further embodiment, the present invention provides a method of treating cancer wherein the cancer is breast cancer, prostate cancer, lung cancer, head and neck cancer, colorectal cancer, pancreatic cancer, gastric cancer, kidney cancer, bladder cancer, melanoma, ovarian cancer, esophageal cancer, soft tissue sarcoma, or hepatocellular carcinoma. In a further embodiment, the present invention provides a method of treating cancer wherein the cancer is breast cancer. In a further embodiment, the present invention provides a method of treating cancer wherein the cancer is prostate cancer. In a further embodiment, the present invention provides a method of treating cancer wherein the cancer is lung cancer. In a further embodiment, the present invention provides a method of treating cancer wherein the cancer is head and neck cancer. In a further embodiment, the present invention provides a method of treating cancer wherein the cancer is colorectal cancer. In a further embodiment, the present invention provides a method of treating cancer wherein the cancer is pancreatic cancer. In a further embodiment, the present invention provides a method of treating cancer wherein the cancer is gastric cancer. In a further embodiment, the present invention provides a method of treating cancer wherein the cancer is kidney cancer. In a further embodiment, the present invention provides a method of treating cancer wherein the cancer is bladder cancer. In a further embodiment, the present invention provides a method of treating cancer wherein the cancer is melanoma. In a further embodiment, the present invention provides a method of treating cancer wherein the cancer is ovarian cancer. In a further embodiment, the present invention provides a method of treating cancer wherein the cancer is esophageal cancer. In a further embodiment, the present invention provides a method of treating cancer wherein the cancer is soft tissue sarcoma. In a further embodiment, the present invention provides a method of treating cancer wherein the cancer is hepatocellular carcinoma.

In a further embodiment, the present invention provides a method of treating cancer wherein the cancer has macrophage infiltration.

In a further embodiment, the present invention provides a method of treating cancer wherein the cancer is a solid tumor.

In a further embodiment, these methods comprise the administration of an effective amount of the CSF-1R antibody and PD-L1 antibody in simultaneous, separate, or sequential combination with one or more anti-tumor agents selected from cisplatin, carboplatin, dacarbazine, liposomal doxorubicin, docetaxel, cyclophosphamide and doxorubicin, navelbine, eribulin, paclitaxel, paclitaxel protein-bound particles for injectable suspension, ixabepilone, capecitabine, FOLFOX (leucovorin, fluorouracil, and oxaliplatin), FOLFIRI (leucovorin, fluorouracil, and irinotecan), and cetuximab.

In some embodiments the present invention provides a combination comprising a first antibody that binds CSF-1R and a second antibody that binds PD-L1, wherein: the first antibody comprises a LC and a HC, wherein the LC comprises a LCVR and the HC comprises a HCVR, and wherein the LCVR comprises light chain complementarity determining regions LCDR1, LCDR2, and LCDR3 consisting of the amino acid sequences RASQGISNALA (SEQ ID NO: 16), DASSLES (SEQ ID NO: 17), and QQFNSYPWT (SEQ ID NO: 18), respectively, and wherein the HCVR comprises heavy chain complementarity determining regions HCDR1, HCDR2, and HCDR3 consisting of the amino acid sequences SYGMH (SEQ ID NO: 13), VIWYDGSNKYYADSVKG (SEQ ID NO: 14), and GDYEVDYGMDV (SEQ ID NO: 15), respectively; and the second antibody comprises a LC and a HC, wherein the LC comprises a LCVR and the HC comprises a HCVR, and wherein the LCVR comprises light chain complementarity determining regions LCDR1, LCDR2, and LCDR3 consisting of the amino acid sequences SGSSSNIGSNTVN (SEQ ID NO: 22), YGNSNRPS (SEQ ID NO: 23), and QSYDSSLSGSV (SEQ ID NO: 24), respectively, and wherein the HCVR comprises heavy chain complementarity determining regions HCDR1, HCDR2, and HCDR3 consisting of the amino acid sequences KASGGTFSSYAIS (SEQ ID NO: 19), GIIPIFGTANYAQKFQG (SEQ ID NO: 20), and ARSPDYSPYYYYGMDV (SEQ ID NO: 21), respectively; for simultaneous, separate, or sequential use in the treatment of cancer.

In some embodiments the present invention provides a combination comprising a first antibody and a second antibody, wherein: the first antibody comprises a LC and a HC, wherein the LC comprises a LCVR and the HC comprises a HCVR, wherein the LCVR has the amino acid sequence given in SEQ ID NO: 2, and the HCVR has the amino acid sequence given in SEQ ID NO: 1; and the second antibody comprises a LC and a HC, wherein the LC comprises a LCVR and the HC comprises a HCVR, wherein the LCVR has the amino acid sequence given in SEQ ID NO: 6, and the HCVR has the amino acid sequence given in SEQ ID NO: 5; for simultaneous, separate, or sequential use in the treatment of cancer.

In a further embodiment, the present invention provides a combination wherein the LC of the first antibody has the amino acid sequence given in SEQ ID NO: 4, and the HC of the first antibody has the amino acid sequence given in SEQ ID NO: 3 for simultaneous, separate, or sequential use in the treatment of cancer.

In a further embodiment, the present invention provides a combination wherein the LC of the second antibody has the amino acid sequence given in SEQ ID NO: 8, and the HC of the second antibody has the amino acid sequence given in SEQ ID NO: 7 for simultaneous, separate, or sequential use in the treatment of cancer.

In a further embodiment, the present invention provides a combination wherein: the first antibody comprises two light chains and two heavy chains, wherein each light chain has the amino acid sequence given in SEQ ID NO: 4, and each heavy chain has the amino acid sequence given in SEQ ID NO: 3; and the second antibody comprises two light chains and two heavy chains, wherein each light chain has the amino acid sequence given in SEQ ID NO: 8, and each heavy chain has the amino acid sequence given in SEQ ID NO: 7; for simultaneous, separate, or sequential use in the treatment of cancer.

In some embodiments the present invention provides a combination comprising a first antibody that binds to human CSF-1R and a second antibody that binds to human PD-1, wherein: the first antibody comprises a LC and a HC, wherein the LC comprises a LCVR and the HC comprises a HCVR, wherein the LCVR has the amino acid sequence given in SEQ ID NO: 2, and the HCVR has the amino acid sequence given in SEQ ID NO: 1; and the second antibody comprises a LC and a HC, wherein the LC comprises a LCVR and the HC comprises a HCVR, wherein the LCVR has the amino acid sequence given in SEQ ID NO: 6, and the HCVR has the amino acid sequence given in SEQ ID NO: 5; for simultaneous, separate, or sequential use in the treatment of cancer.

In a further embodiment, the present invention provides a combination wherein: the first antibody that binds to human CSF-1R comprises a LC and a HC, wherein the LC has the amino acid sequence given in SEQ ID NO: 4, and the HC has the amino acid sequence given in SEQ ID NO: 3; and the second antibody that binds to human PD-L1 comprises a LC and a HC, wherein the LC has the amino acid sequence given in SEQ ID NO: 8, and the HC has the amino acid sequence given in SEQ ID NO: 7; for simultaneous, separate, or sequential use in the treatment of cancer.

In a further embodiment, the present invention provides a combination wherein: the first antibody that binds to human CSF-1R comprises two light chains and two heavy chains, wherein each light chain has the amino acid sequence given in SEQ ID NO: 4, and each heavy chain has the amino acid sequence given in SEQ ID NO: 3; and the second antibody that binds to human PD-L1 comprises two light chains and two heavy chains, wherein each light chain has the amino acid sequence given in SEQ ID NO: 8, and each heavy chain has the amino acid sequence given in SEQ ID NO: 7; for simultaneous, separate, or sequential use in the treatment of cancer.

In some embodiments the present invention provides an antibody that binds human PD-L1, comprising a LC and a HC, wherein the LC comprises a LCVR and the HC comprises a HCVR, and wherein the LCVR comprises light chain complementarity determining regions LCDR1, LCDR2, and LCDR3 consisting of the amino acid sequences SGSSSNIGSNTVN (SEQ ID NO: 22), YGNSNRPS (SEQ ID NO: 23), and QSYDSSLSGSV (SEQ ID NO: 24), respectively, and wherein the HCVR comprises heavy chain complementarity determining regions HCDR1, HCDR2, and HCDR3 consisting of the amino acid sequences KASGGTFSSYAIS (SEQ ID NO: 19), GIIPIFGTANYAQKFQG (SEQ ID NO: 20), and ARSPDYSPYYYYGMDV (SEQ ID NO: 21), respectively, for use in simultaneous, separate, or sequential combination with an antibody that binds human CSF-1R, comprising a LC and a HC, wherein the LC comprises a LCVR and the HC comprises a HCVR, and wherein the LCVR comprises light chain complementarity determining regions LCDR1, LCDR2, and LCDR3 consisting of the amino acid sequences RASQGISNALA (SEQ ID NO: 16), DASSLES (SEQ ID NO: 17), and QQFNSYPWT (SEQ ID NO: 18), respectively, and wherein the HCVR comprises heavy chain complementarity determining regions HCDR1, HCDR2, and HCDR3 consisting of the amino acid sequences SYGMH (SEQ ID NO: 13), VIWYDGSNKYYADSVKG (SEQ ID NO: 14), and GDYEVDYGMDV (SEQ ID NO: 15), respectively, in the treatment of cancer.

In some embodiments the present invention provides an antibody that binds human PD-L1, comprising a LC and a HC, wherein the LC comprises a LCVR and the HC comprises a HCVR, wherein the LCVR has the amino acid sequence given in SEQ ID NO: 6, and the HCVR has the amino acid sequence given in SEQ ID NO: 5, for use in simultaneous, separate, or sequential combination with an antibody that binds human CSF-1R, comprising a LC and a HC, wherein the LC comprises a LCVR and the HC comprises a HCVR, wherein the LCVR has the amino acid sequence given in SEQ ID NO: 2, and the HCVR has the amino acid sequence given in SEQ ID NO: 1, in the treatment of cancer.

In a further embodiment, the present invention provides an antibody that binds human PD-L1, comprising a LC and a HC, wherein the LC has the amino acid sequence given in SEQ ID NO: 8, and the HC has the amino acid sequence given in SEQ ID NO: 7, for use in simultaneous, separate, or sequential combination with an antibody that binds human CSF-1R, comprising a LC and a HC, wherein the LC has the amino acid sequence given in SEQ ID NO: 4, and the HC has the amino acid sequence given in SEQ ID NO: 3, in the treatment of cancer.

In a further embodiment, the present invention provides an antibody that binds human PD-L1, comprising two light chains and two heavy chains, wherein each light chain has the amino acid sequence given in SEQ ID NO: 8, and each heavy chain has the amino acid sequence given in SEQ ID NO: 7, for use in simultaneous, separate, or sequential combination with an antibody that binds human CSF-1R, comprising two light chains and two heavy chains, wherein each light chain has the amino acid sequence given in SEQ ID NO: 4, and each heavy chain has the amino acid sequence given in SEQ ID NO: 3, in the treatment of cancer.

In some embodiments the present invention provides an antibody that binds human CSF-1R, comprising a LC and a HC, wherein the LC comprises a LCVR and the HC comprises a HCVR, and wherein the LCVR comprises light chain complementarity determining regions LCDR1, LCDR2, and LCDR3 consisting of the amino acid sequences RASQGISNALA (SEQ ID NO: 16), DASSLES (SEQ ID NO: 17), and QQFNSYPWT (SEQ ID NO: 18), respectively, and wherein the HCVR comprises heavy chain complementarity determining regions HCDR1, HCDR2, and HCDR3 consisting of the amino acid sequences SYGMH (SEQ ID NO: 13), VIWYDGSNKYYADSVKG (SEQ ID NO: 14), and GDYEVDYGMDV (SEQ ID NO: 15), respectively, for use in simultaneous, separate, or sequential combination with an antibody that binds human PD-L1, comprising a LC and a HC, wherein the LC comprises a LCVR and the HC comprises a HCVR, and wherein the LCVR comprises light chain complementarity determining regions LCDR1, LCDR2, and LCDR3 consisting of the amino acid sequences SGSSSNIGSNTVN (SEQ ID NO: 22), YGNSNRPS (SEQ ID NO: 23), and QSYDSSLSGSV (SEQ ID NO: 24), respectively, and wherein the HCVR comprises heavy chain complementarity determining regions HCDR1, HCDR2, and HCDR3 consisting of the amino acid sequences KASGGTFSSYAIS (SEQ ID NO: 19), GIIPIFGTANYAQKFQG (SEQ ID NO: 20), and ARSPDYSPYYYYGMDV (SEQ ID NO: 21), respectively, in the treatment of cancer.

In some embodiments the present invention provides an antibody that binds human CSF-1R, comprising a LC and a HC, wherein the LC comprises a LCVR and the HC comprises a HCVR, wherein the LCVR has the amino acid sequence given in SEQ ID NO: 2, and the HCVR has the amino acid sequence given in SEQ ID NO: 1, for use in simultaneous, separate, or sequential combination with an antibody that binds human PD-L1, comprising a LC and a HC, wherein the LC comprises a LCVR and the HC comprises a HCVR, wherein the LCVR has the amino acid sequence given in SEQ ID NO: 6, and the HCVR has the amino acid sequence given in SEQ ID NO: 5, in the treatment of cancer.

In some embodiments the present invention provides an antibody that binds human CSF-1R, comprising a LC and a HC, wherein the LC has the amino acid sequence given in SEQ ID NO: 4, and the HC has the amino acid sequence given in SEQ ID NO: 3, for use in simultaneous, separate, or sequential combination with an antibody that binds human PD-L1, comprising a LC and a HC, wherein the LC has the amino acid sequence given in SEQ ID NO: 8, and the HC has the amino acid sequence given in SEQ ID NO: 7, in the treatment of cancer.

In a further embodiment, the present invention provides an antibody that binds human CSF-1R, comprising two light chains and two heavy chains, wherein each light chain has the amino acid sequence given in SEQ ID NO: 4, and each heavy chain has the amino acid sequence given in SEQ ID NO: 3, for use in simultaneous, separate, or sequential combination with an antibody that binds human PD-L1, comprising two light chains and two heavy chains, wherein each light chain has the amino acid sequence given in SEQ ID NO: 8, and each heavy chain has the amino acid sequence given in SEQ ID NO: 7, in the treatment of cancer.

In a preferred embodiment, the cancer is breast cancer, prostate cancer, lung cancer, head and neck cancer, colorectal cancer, pancreatic cancer, gastric cancer, kidney cancer, bladder cancer, melanoma, ovarian cancer, esophageal cancer, soft tissue sarcoma, or hepatocellular carcinoma. In a still further preferred embodiment, the cancer is breast cancer. In a still further preferred embodiment, the cancer is prostate cancer. In a still further preferred embodiment, the cancer is lung cancer. In a still further preferred embodiment, the cancer is head and neck cancer. In a still further preferred embodiment, the cancer is colorectal cancer. In a still further preferred embodiment, the cancer is pancreatic cancer. In a still further preferred embodiment, the cancer is gastric cancer. In a still further preferred embodiment, the cancer is kidney cancer. In a still further preferred embodiment, the cancer is bladder cancer. In a still further preferred embodiment, the cancer is melanoma. In a still further preferred embodiment, the cancer is ovarian cancer. In a still further preferred embodiment, the cancer is esophageal cancer. In a still further preferred embodiment, the cancer is soft tissue sarcoma. In a still further preferred embodiment, the cancer is hepatocellular carcinoma.

In a still further preferred embodiment, the cancer has macrophage infiltration.

In a still further preferred embodiment, the cancer is a solid tumor.

In a further embodiment, the present invention provides a combination of the present invention for use in simultaneous, separate, or sequential combination with one or more anti-tumor agents. In a further embodiment, the present invention provides the CSF-1R antibody and PD-L antibody for use in simultaneous, separate, or sequential combination with one or more anti-tumor agents selected from the group consisting of cisplatin, carboplatin, dacarbazine, liposomal doxorubicin, docetaxel, cyclophosphamide and doxorubicin, navelbine, eribulin, paclitaxel, paclitaxel protein-bound particles for injectable suspension, ixabepilone, capecitabine, FOLFOX (leucovorin, fluorouracil, and oxaliplatin), FOLFIRI (leucovorin, fluorouracil, and irinotecan), and cetuximab, in the treatment of cancer.

In some embodiments the present invention provides the use of an antibody that binds human PD-L1, comprising a LC and a HC, wherein the LC comprises a LCVR and the HC comprises a HCVR, and wherein the LCVR comprises light chain complementarity determining regions LCDR1, LCDR2, and LCDR3 consisting of the amino acid sequences SGSSSNIGSNTVN (SEQ ID NO: 22), YGNSNRPS (SEQ ID NO: 23), and QSYDSSLSGSV (SEQ ID NO: 24), respectively, and wherein the HCVR comprises heavy chain complementarity determining regions HCDR1, HCDR2, and HCDR3 consisting of the amino acid sequences KASGGTFSSYAIS (SEQ ID NO: 19), GIIPIFGTANYAQKFQG (SEQ ID NO: 20), and ARSPDYSPYYYYGMDV (SEQ ID NO: 21), respectively, in the manufacture of a medicament for the treatment of cancer, wherein the antibody that binds human PD-L is administered simultaneously, separately, or sequentially with an antibody that binds human CSF-1R, comprising a LC and a HC, wherein the LC comprises a LCVR and the HC comprises a HCVR, and wherein the LCVR comprises light chain complementarity determining regions LCDR1, LCDR2, and LCDR3 consisting of the amino acid sequences RASQGISNALA (SEQ ID NO: 16), DASSLES (SEQ ID NO: 17), and QQFNSYPWT (SEQ ID NO: 18), respectively, and wherein the HCVR comprises heavy chain complementarity determining regions HCDR1, HCDR2, and HCDR3 consisting of the amino acid sequences SYGMH (SEQ ID NO: 13), VIWYDGSNKYYADSVKG (SEQ ID NO: 14), and GDYEVDYGMDV (SEQ ID NO: 15), respectively.

In some embodiments the present invention provides the use of an antibody that binds human PD-L1, comprising a LC and a HC, wherein the LC comprises a LCVR and the HC comprises a HCVR, wherein the LCVR has the amino acid sequence given in SEQ ID NO: 6, and the HCVR has the amino acid sequence given in SEQ ID NO: 5, in the manufacture of a medicament for the treatment of cancer, wherein the antibody that binds human PD-L1 is administered simultaneously, separately, or sequentially with an antibody that binds human CSF-1R, comprising a LC and a HC, wherein the LC comprises a LCVR and the HC comprises a HCVR, wherein the LCVR has the amino acid sequence given in SEQ ID NO: 2, and the HCVR has the amino acid sequence given in SEQ ID NO: 1.

In a further embodiment, the present invention provides the use of an antibody that binds human PD-L1, comprising a LC and a HC, wherein the LC has the amino acid sequence given in SEQ ID NO: 8, and the HC has the amino acid sequence given in SEQ ID NO: 7, in the manufacture of a medicament for the treatment of cancer, wherein the antibody that binds human PD-L1 is administered simultaneously, separately, or sequentially with an antibody that binds human CSF-1R, comprising a LC and a HC, wherein the LC has the amino acid sequence given in SEQ ID NO: 4, and the HC has the amino acid sequence given in SEQ ID NO: 3.

In a further embodiment, the present invention provides the use of an antibody that binds human PD-L1, comprising two light chains and two heavy chains, wherein each light chain has the amino acid sequence given in SEQ ID NO: 8, and each heavy chain has the amino acid sequence given in SEQ ID NO: 7, in the manufacture of a medicament, wherein the antibody that binds human PD-L1 is administered simultaneously, separately, or sequentially with an antibody that binds human CSF-1R, comprising two light chains and two heavy chains, wherein each light chain has the amino acid sequence given in SEQ ID NO: 4, and each heavy chain has the amino acid sequence given in SEQ ID NO: 3.

Preferably, the medicament is administered simultaneously, separately, or sequentially with one or more anti-tumor agents selected from the group consisting of cisplatin, carboplatin, dacarbazine, liposomal doxorubicin, docetaxel, cyclophosphamide and doxorubicin, navelbine, eribulin, paclitaxel, paclitaxel protein-bound particles for injectable suspension, ixabepilone, capecitabine, FOLFOX (leucovorin, fluorouracil, and oxaliplatin), FOLFIRI (leucovorin, fluorouracil, and irinotecan), and cetuximab.

In some embodiments the present invention provides the use of an antibody that binds human CSF-1R, comprising a LC and a HC, wherein the LC comprises a LCVR and the HC comprises a HCVR, and wherein the LCVR comprises light chain complementarity determining regions LCDR1, LCDR2, and LCDR3 consisting of the amino acid sequences RASQGISNALA (SEQ ID NO: 16), DASSLES (SEQ ID NO: 17), and QQFNSYPWT (SEQ ID NO: 18), respectively, and wherein the HCVR comprises heavy chain complementarity determining regions HCDR1, HCDR2, and HCDR3 consisting of the amino acid sequences SYGMH (SEQ ID NO: 13), VIWYDGSNKYYADSVKG (SEQ ID NO: 14), and GDYEVDYGMDV (SEQ ID NO: 15), respectively, in the manufacture of a medicament for the treatment of cancer, wherein the antibody that binds human CSF-1R is administered simultaneously, separately, or sequentially with an antibody that binds human PD-L1, comprising a LC and a HC, wherein the LC comprises a LCVR and the HC comprises a HCVR, and wherein the LCVR comprises light chain complementarity determining regions LCDR1, LCDR2, and LCDR3 consisting of the amino acid sequences SGSSSNIGSNTVN (SEQ ID NO: 22), YGNSNRPS (SEQ ID NO: 23), and QSYDSSLSGSV (SEQ ID NO: 24), respectively, and wherein the HCVR comprises heavy chain complementarity determining regions HCDR1, HCDR2, and HCDR3 consisting of the amino acid sequences KASGGTFSSYAIS (SEQ ID NO: 19), GIIPIFGTANYAQKFQG (SEQ ID NO: 20), and ARSPDYSPYYYYGMDV (SEQ ID NO: 21).

In some embodiments the present invention provides the use of an antibody that binds human CSF-1R, comprising a LC and a HC, wherein the LC comprises a LCVR and the HC comprises a HCVR, wherein the LCVR has the amino acid sequence given in SEQ ID NO: 2, and the HCVR has the amino acid sequence given in SEQ ID NO: 1, in the manufacture of a medicament for the treatment of cancer, wherein the antibody that binds human CSF-1R is administered simultaneously, separately, or sequentially with an antibody that binds human PD-L1, comprising a LC and a HC, wherein the LC comprises a LCVR and the HC comprises a HCVR, wherein the LCVR has the amino acid sequence given in SEQ ID NO: 6, and the HCVR has the amino acid sequence given in SEQ ID NO: 5.

In some embodiments the present invention provides the use of an antibody that binds human CSF-1R, comprising a LC and a HC, wherein the LC has the amino acid sequence given in SEQ ID NO: 4, and the HC has the amino acid sequence given in SEQ ID NO: 3, in the manufacture of a medicament for the treatment of cancer, wherein the antibody that binds CSF-1R is administered simultaneously, separately, or sequentially with an antibody that binds human PD-L, comprising a LC and a HC, wherein the LC has the amino acid sequence given in SEQ ID NO: 8, and the HC has the amino acid sequence given in SEQ ID NO: 7.

In a further embodiment, the present invention provides the use of an antibody that binds human CSF-1R, comprising two light chains and two heavy chains, wherein each light chain has the amino acid sequence given in SEQ ID NO: 4, and each heavy chain has the amino acid sequence given in SEQ ID NO: 3, in the manufacture of a medicament for the treatment of cancer, wherein the antibody that binds CSF-1R is administered simultaneously, separately, or sequentially with an antibody that binds human PD-L1, comprising two light chains and two heavy chains, wherein each light chain has the amino acid sequence given in SEQ ID NO: 8, and each heavy chain has the amino acid sequence given in SEQ ID NO: 7.

Preferably, the medicament is also administered simultaneously, separately, or sequentially with one or more anti-tumor agents selected from the group consisting of cisplatin, carboplatin, dacarbazine, liposomal doxorubicin, docetaxel, cyclophosphamide and doxorubicin, navelbine, eribulin, paclitaxel, paclitaxel protein-bound particles for injectable suspension, ixabepilone, capecitabine, FOLFOX (leucovorin, fluorouracil, and oxaliplatin), FOLFIRI (leucovorin, fluorouracil, and irinotecan), and cetuximab.

In a preferred embodiment, the cancer is breast cancer, prostate cancer, lung cancer, head and neck cancer, colorectal cancer, pancreatic cancer, gastric cancer, kidney cancer, bladder cancer, melanoma, ovarian cancer, esophageal cancer, soft tissue sarcoma, or hepatocellular carcinoma. In a still further preferred embodiment, the cancer is breast cancer. In a still further preferred embodiment, the cancer is prostate cancer. In a still further preferred embodiment, the cancer is lung cancer. In a still further preferred embodiment, the cancer is head and neck cancer. In a still further preferred embodiment, the cancer is colorectal cancer. In a still further preferred embodiment, the cancer is pancreatic cancer. In a still further preferred embodiment, the cancer is gastric cancer. In a still further preferred embodiment, the cancer is kidney cancer. In a still further preferred embodiment, the cancer is bladder cancer. In a still further preferred embodiment, the cancer is melanoma In a still further preferred embodiment, the cancer is ovarian cancer. In a still further preferred embodiment, the cancer is esophageal cancer. In a still further preferred embodiment, the cancer is soft tissue sarcoma. In a still further preferred embodiment, the cancer is hepatocellular carcinoma.

In a still further preferred embodiment, the cancer has macrophage infiltration.

In a still further preferred embodiment, the cancer is a solid tumor.

In a further embodiment, the present invention provides the use of a combination of the present invention in the manufacture of a medicament for the treatment of cancer wherein said medicament is to be administered simultaneously, separately, or sequentially with one or more anti-tumor agents selected from the group consisting of cisplatin, carboplatin, dacarbazine, liposomal doxorubicin, docetaxel, cyclophosphamide and doxorubicin, navelbine, eribulin, paclitaxel, paclitaxel protein-bound particles for injectable suspension, ixabepilone, capecitabine, FOLFOX (leucovorin, fluorouracil, and oxaliplatin), FOLFIRI (leucovorin, fluorouracil, and irinotecan), and cetuximab.

In some embodiments the present invention provides a kit comprising a first antibody that binds human CSF-1R and a second antibody that binds human PD-L1, wherein: the first antibody comprises a LC and a HC, wherein the LC comprises a LCVR and the HC comprises a HCVR, and wherein the LCVR comprises light chain complementarity determining regions LCDR1, LCDR2, and LCDR3 consisting of the amino acid sequences RASQGISNALA (SEQ ID NO: 16), DASSLES (SEQ ID NO: 17), and QQFNSYPWT (SEQ ID NO: 18), respectively, and wherein the HCVR comprises heavy chain complementarity determining regions HCDR1, HCDR2, and HCDR3 consisting of the amino acid sequences SYGMH (SEQ ID NO: 13), VIWYDGSNKYYADSVKG (SEQ ID NO: 14), and GDYEVDYGMDV (SEQ ID NO: 15), respectively; and the second antibody comprises a LC and a HC, wherein the LC comprises a LCVR and the HC comprises a HCVR, and wherein the LCVR comprises light chain complementarity determining regions LCDR1, LCDR2, and LCDR3 consisting of the amino acid sequences SGSSSNIGSNTVN (SEQ ID NO: 22), YGNSNRPS (SEQ ID NO: 23), and QSYDSSLSGSV (SEQ ID NO: 24), respectively, and wherein the HCVR comprises heavy chain complementarity determining regions HCDR1, HCDR2, and HCDR3 consisting of the amino acid sequences KASGGTFSSYAIS (SEQ ID NO: 19), GIIPIFGTANYAQKFQG (SEQ ID NO: 20), and ARSPDYSPYYYYGMDV (SEQ ID NO: 21), respectively.

In some embodiments the present invention provides a kit comprising a first antibody and a second antibody, wherein: the first antibody comprises a LC and a HC, wherein the LC comprises a LCVR and the HC comprises a HCVR, wherein the LCVR has the amino acid sequence given in SEQ ID NO: 2, and the HCVR has the amino acid sequence given in SEQ ID NO: 1; and the second antibody comprises a LC and a HC, wherein the LC comprises a LCVR and the HC comprises a HCVR, wherein the LCVR has the amino acid sequence given in SEQ ID NO: 6, and the HCVR has the amino acid sequence given in SEQ ID NO: 5.

In a further embodiment, the present invention provides a kit wherein the LC of the first antibody has the amino acid sequence given in SEQ ID NO: 4, and the HC of the first antibody has the amino acid sequence given in SEQ ID NO: 3. In a further embodiment, the present invention provides a kit wherein the LC of the second antibody has the amino acid sequence given in SEQ ID NO: 8, and the HC of the second antibody has the amino acid sequence given in SEQ ID NO: 7.

In a further embodiment, the present invention provides a kit wherein: the first antibody comprises two light chains and two heavy chains, wherein each light chain has the amino acid sequence given in SEQ ID NO: 4, and each heavy chain has the amino acid sequence given in SEQ ID NO: 3; and the second antibody comprises two light chains and two heavy chains, wherein each light chain has the amino acid sequence given in SEQ ID NO: 8, and each heavy chain has the amino acid sequence given in SEQ ID NO: 7.

In some embodiments the present invention provides a kit comprising a first antibody that binds to human CSF-1R and a second antibody that binds to human PD-L1, wherein: the first antibody comprises a LC and a HC, wherein the LC comprises a LCVR and the HC comprises a HCVR, wherein the LCVR has the amino acid sequence given in SEQ ID NO: 2, and the HCVR has the amino acid sequence given in SEQ ID NO: 1; and the second antibody comprises a LC and a HC, wherein the LC comprises a LCVR and the HC comprises a HCVR, wherein the LCVR has the amino acid sequence given in SEQ ID NO: 6, and the HCVR has the amino acid sequence given in SEQ ID NO: 5.

In a further embodiment, the present invention provides a kit wherein: the first antibody that binds to human CSF-1R comprises two light chains and two heavy chains, wherein each light chain has the amino acid sequence given in SEQ ID NO: 4, and each heavy chain has the amino acid sequence given in SEQ ID NO: 3; and the second antibody that binds to human PD-L1 comprises two light chains and two heavy chains, wherein each light chain has the amino acid sequence given in SEQ ID NO: 8, and each heavy chain has the amino acid sequence given in SEQ ID NO: 7.

Anti-CSF-1R antibody, Antibody 1, is a recombinant IgG1 human monoclonal antibody targeting human CSF-1R. CSF-1R, as specified herein, includes either variation of CSF-1R as disclosed in SEQ ID NOS: 15 and 16 of U.S. Pat. No. 8,263,079. Antibody 1 and methods of making and using this antibody including for the treatment of neoplastic diseases such as solid tumors are disclosed in U.S. Pat. No. 8,263,079. Furthermore, clinical study of Antibody 1 is ongoing in two clinical trials (NCT01346358 and NCT02265536).

Antibody 1 comprises a LC having the amino acid sequence given in SEQ ID NO: 4, and a HC having the amino acid sequence given in SEQ ID NO: 3.

Anti-PD-L1 antibody, Antibody A, is a recombinant IgG1 human monoclonal antibody targeting human PD-L1. PD-L1 as disclosed in SEQ ID NO: 1 in U.S. 62/209,056. Antibody A and methods of making and using this antibody including for the treatment of cancer are disclosed in U.S. 62/209,056.

Antibody A comprises a light chain (LC) having the amino acid sequence given in SEQ ID NO: 8, and a heavy chain (HC) having the amino acid sequence given in SEQ ID NO: 7.

As used herein, the terms “treating,” “to treat,” or “treatment” refers to restraining, slowing, stopping, reducing, or reversing the progression or severity of an existing symptom, disorder, condition, or disease.

As used herein, the term “patient” refers to a mammal, preferably a human.

As used herein, the terms “cancer” and “cancerous” refer to or describe the physiological condition in patients that is typically characterized by unregulated cell growth. Included in this definition are benign and malignant cancers.

As used herein, the term “kit” refers to a package comprising at least two separate containers, wherein a first container contains an anti-CSF-1R antibody and a second container contains an anti-PD-L1 antibody. As used herein, the term “kit” may also refer to a package comprising at least two separate containers, wherein a first container contains an anti-PD-L1 antibody and a second container contains an anti-CSF-1R antibody. A “kit” may also include instructions to administer all or a portion of the contents of these first and second containers to a cancer patient.

The efficacy of the combination treatment of the invention can be measured by various endpoints commonly used in evaluating cancer treatments, including but not limited to, tumor regression, tumor weight or size shrinkage, time to progression, duration of survival, progression free survival, overall response rate, duration of response, and quality of life. The therapeutic agents used in the invention may cause inhibition of metastatic spread without shrinkage of the primary tumor, or may simply exert a tumoristatic effect. Because the invention relates to the use of a combination of unique anti-tumor agents, novel approaches to determining efficacy of any particular combination therapy of the present invention can be optionally employed, including, for example, measurement of plasma or urinary markers of angiogenesis and measurement of response through radiological imaging.

As used herein, the term “effective amount” or “effective dose” refers to the amount of an antibody of the present invention or pharmaceutical composition comprising an antibody of the present invention that will elicit the biological or medical response of or desired therapeutic effect on a tissue, system, animal, mammal or human that is being sought by the researcher, medical doctor, or other clinician. An effective amount of the antibody may vary according to factors such as the disease state, age, sex, and weight of the individual, and the ability of the antibody to elicit a desired response in the individual. An effective amount is also one in which any toxic or detrimental effect of the antibody is outweighed by the therapeutically beneficial effects.

Dosage regimens may be adjusted to provide the optimum desired response (e.g., a therapeutic response). Treatment dosages may be titrated using routine methods known to those of skill in the art to optimize safety and efficacy. Dosing schedules, for intravenous (i.v.) or non-intravenous administration, localized or systemic, or combinations thereof, will typically range from a single bolus dosage or continuous infusion to multiple administrations per day (e.g., every 4-6 hours), or as indicated by the treating physician and the patient's condition.

An exemplary, non-limiting range for a therapeutically effective amount of Antibody 1 is 0.1-5 mg/kg, and preferably 0.3-2 mg/kg, and more preferably 0.6-1.25 mg/kg. These non-limiting ranges can be given once a week or twice a week. An exemplary, non-limiting range for a therapeutically effective amount of Antibody 1 is a 50 mg-200 mg flat dose once a week, and more preferably 100-150 mg flat dose once a week. Dosing amounts and frequencies will be determined by the physicians treating the patient and may include doses from less than 1 mg/kg to over 100 mg/kg given daily, three times per week, weekly, once every two weeks, or less often. It should be noted, however, that the present invention is not limited to any particular dose.

An exemplary, non-limiting range for a therapeutically effective amount of Antibody A is 0.1-10 mg/kg. These non-limiting ranges can be given every week, every two weeks, every three weeks, or once a month. An exemplary, non-limiting range for a therapeutically effective amount of Antibody 1 is a 70 mg-700 mg flat dose once every two weeks. Dosing amounts and frequencies will be determined by the physicians treating the patient and may include doses from less than 0.1 mg/kg to over 10 mg/kg. It should be noted, however, that the present invention is not limited to any particular dose.

In the combination of the present invention in some instances dosage levels below the lower limit of the aforesaid ranges for Antibody 1 and Antibody A, may be more than adequate, while in other cases smaller or still larger doses may be employed with acceptable side effects. Dosing amounts and frequencies will be determined by the physicians treating the patient. It should be noted, however, that the present invention is not limited to any particular dose.

“Durable complete regression” is a reduction in tumor burden that results in no evaluable disease present, without recurrence of disease during the observation period which was greater than 30 days past the survival of last untreated subject.

“Control of tumor burden”, or “disease control” is the slowing, stopping or regression of tumor growth that does not result in complete tumor regression, but which results in prolonged survival causing clinical benefit for a subject compared to, or superior to other available treatments.

Each antibody of the present invention, or pharmaceutical compositions comprising the same, may be administered by parenteral routes (e.g., subcutaneous and intravenous). Each antibody of the present invention may be administered to a patient alone with pharmaceutically acceptable carriers, diluents, or excipients in single or multiple doses. Pharmaceutical compositions of the present invention can be prepared by methods well known in the art (e.g., Remington: The Science and Practice of Pharmacy, 22^{nd} ed. (2012), A. Loyd et al., Pharmaceutical Press) and comprise an antibody, as disclosed herein, and one or more pharmaceutically acceptable carriers, diluents, or excipients.

As used herein, the phrase “in combination with” refers to the administration of an anti-CSF-1R antibody, with an anti-PD-L1 antibody simultaneously. As used herein, the phrase “in combination with” also refers to the administration of an anti-CSF-1R antibody, with an anti-PD-L1 antibody sequentially in any order. As used herein, the phrase “in combination with” also refers to the administration of an anti-CSF-1R antibody, with an anti-PD-L1 antibody in any combination thereof. An anti-CSF-1R antibody can be administered prior to administration of an anti-PD-L1 antibody. An anti-CSF-1R antibody can be administered at the same time as administration of an anti-PD-L1 antibody. An anti-CSF-1R antibody can be administered subsequent to administration of an anti-PD-L1 antibody. An anti-CSF-1R antibody can be administered prior to, at the same time as, or subsequent to administration of an anti-PD-L1 antibody, or in some combination thereof.

Where an anti-PD-L1 antibody is administered at repeated intervals (e.g. during a standard course of treatment), an anti-CSF-1R antibody can be administered prior to each administration of an anti-PD-L1 antibody. Where an anti-PD-L1 antibody is administered at repeated intervals (e.g. during a standard course of treatment), an anti-CSF-1R antibody can be administered at the same time as each administration of an anti-PD-L1 antibody. Where an anti-PD-L1 antibody is administered at repeated intervals (e.g. during a standard course of treatment), an anti-CSF-1R antibody can be administered subsequent to each administration of an anti-PD-L1 antibody. Where an anti-PD-L1 antibody is administered at repeated intervals (e.g. during a standard course of treatment), an anti-CSF-1R antibody can be administered prior to, at the same time as, or subsequent to, each administration of an anti-PD-L1 antibody or some combination thereof. Where an anti-PD-L1 antibody is administered at repeated intervals (e.g. during a standard course of treatment), an anti-CSF-1R antibody can be administered at different intervals in relation to therapy with an anti-PD-L1 antibody. Where an anti-PD-L1 antibody is administered at repeated intervals (e.g. during a standard course of treatment), an anti-CSF-1R antibody can be administered in a single or series of dose(s) prior to, at any time during, or subsequent to the course of treatment with an anti-PD-L1 antibody. Where an anti-PD-L1 antibody is administered at repeated intervals (e.g. during a standard course of treatment), an anti-CSF-1R antibody can be administered in a single dose prior to, at any time during, or subsequent to the course of treatment with an anti-PD-L1 antibody. Where an anti-PD-L1 antibody is administered at repeated intervals (e.g. during a standard course of treatment), an anti-CSF-1R antibody can be administered in a single dose prior to the course of treatment with an anti-PD-L1 antibody. Where an anti-PD-L1 antibody is administered at repeated intervals (e.g. during a standard course of treatment), an anti-CSF-1R antibody can be administered in a single dose at any time during the course of treatment with an anti-PD-L1 antibody. Where an anti-PD-L1 antibody is administered at repeated intervals (e.g. during a standard course of treatment), an anti-CSF-1R antibody can be administered in a single dose subsequent to the course of treatment with an anti-PD-L1 antibody. Where an anti-PD-L1 antibody is administered at repeated intervals (e.g. during a standard course of treatment), an anti-CSF-1R antibody can be administered in a series of doses prior to the course of treatment with an anti-PD-L1 antibody. Where an anti-PD-L1 antibody is administered at repeated intervals (e.g. during a standard course of treatment), an anti-CSF-1R antibody can be administered in a series of doses subsequent to the course of treatment with an anti-PD-L1 antibody. Where an anti-PD-L1 antibody is administered at repeated intervals (e.g. during a standard course of treatment), an anti-CSF-1R antibody can be administered in a series of doses subsequent to the course of treatment with an anti-PD-L1 antibody.

This invention is further illustrated by the following non-limiting examples. Antibody 1 can be made, for example, according to the disclosure in U.S. Pat. No. 8,263,079. Antibody A can be made, for example, according to the disclosure in U.S. 62/209,056.

### EXAMPLE 1

### Binding Kinetics and Affinity

The kinetics and equilibrium dissociation constant (K_{D}) for human PD-L1 is determined for anti-PD-L1 antibodies of the present invention using surface plasmon resonance (Biacore).

Immobilization of anti-PD-L1 antibodies of the present invention as ligand on to sensor chip surface is performed at 25° C. Soluble human PD-L1-Fc fusion protein is injected as analyte at concentrations ranging from 0.0123 nM-9 nM. The analysis is performed at 37° C. The contact time for each sample is 180 sec at 30 μl/min. The dissociation time was 240-1500 seconds. The immobilized surface is regenerated for 18 seconds with 0.95 M NaCl/25 mM NaOH at 30 l/min, and then stabilized for 30 seconds. Binding kinetics are analyzed using the Biacore T200 Evaluation software (Version 3.0). Data are referenced to a blank flow cell, and the data are fit to a 1:1 binding model.

In experiments performed essentially as described in this assay, Antibody A binds to human PD-L1 with a K_{D} of 82 pM.

The kinetics and equilibrium dissociation constant (K_{D}) for human CSF-1R is determined for anti-CSF-1R antibodies of the present invention using surface plasmon resonance (Biacore).

Measure the binding kinetics of anti-CSF-1R antibodies of the present invention to CSF-1R-Fc at 25° C. on a Biacore® 2000 SPR Biosensor (GE Healthcare). Immobilized soluble CSF-1R-Fc fusion protein (concentration of 10 μg/mL and pH 5), ranging from 395 to 1200 response units, on a CM5 chip using the standard amine coupling protocol. Use HBS-EP (0.01 M HEPES (pH 7.4), 0.15 mM NaCl, and 3 mM EDTA, 0.005% v/v Surfactant P20) as a running buffer during binding affinity measurements. Perform interaction analyses as the antibodies in solution are injected at concentrations ranging from 1.5-100 nM over the prepared surface of the CM5 sensor chip. Inject the antibodies over 3 minutes for binding and allow to dissociate for 15 minutes. Perform regeneration of the immobilized protein by a 10 μL/min injection of 20 mM HCL. Use BIAevaluation version 4.1 software to determine the Kₐ (kₒₙ) and K_{d} (k_{off}) of the complex formation by simultaneous global fitting of the data to a 1:1 Langmuir model. Calculate the equilibrium association constant (K_{A}) from the ratio of 1/K_{D} measured in Molar (1/M).

Calculate the equilibrium dissociation constant (K_{D}) from the ratio of rate constants K_{d}/Kₐ measured in Molar (M).
The average Kₐ, K_{d}, and K_{D} values for multiple Biacore® analyses for Antibody 1 with human CSF-1R are summarized in Table 1.

TABLE 1
Binding Kinetics of Antibody to Recombinant Human CSF-1R

| | Kₐ (1/Ms) | K_{d} (1/s) | K_{D} |
|---|---|---|---|
| Antibody | Kₒₙ | K_{off} | (M) |
| Antibody 1 | 3.7 × 10⁵ | 3.3 × 10⁻⁴ | 8.0 × 10⁻¹⁰ |

### EXAMPLE 2

Efficacy Study of CSF-1R Antibody in Combination with PD-L1 Antibody in CT26 Syngeneic Tumor Model

To determine the efficacy of CSF-1R antibody in combination with PD-L1 antibody, monotherapy and combinations are tested in a CT26 syngenic tumor model, which is a model that is considered to be a macrophage infiltration model.

CT26 is a murine model, accordingly, the CSF-1R antibody and the PD-L1 antibody used in the CT26 model must each bind their respective mouse target protein. Neither Antibody 1 nor Antibody A are cross-reactive to the murine target protein. Accordingly, for CSF-1R, CS7 was used as a surrogate antibody for Antibody 1. Both Antibody 1 and CS7 bind domains 1 and 2 of CSF-1R and so block ligand binding. By blocking ligand binding, both Antibody 1 and CS7 block downstream activation of the receptor preventing phosphorylation of the receptor. Preventing phosphorylation prevents activation of the CSF-1R receptor, in vitro treatment by Antibody 1 and CS7 causes reduced human and murine macrophage viability in vitro respectively. Both Antibody 1 and CS7 reduce macrophage migration in vitro. When dosed in vivo, both Antibody 1 and CS7 and increase the level of circulating CSF-1.

CS7 is a rat anti-mouse CSF-1R antibody (generated from a hybridoma). The HCVR of CS7 is SEQ ID NO: 9, and the LCVR of CS7 is SEQ ID NO: 10.

For PD-L, anti-murine PD-L1 clone, 178G7, is used as a surrogate antibody for Antibody A. Both 178G7 and Antibody A block PD-1 binding to PD-L1. Both 178G7 and Antibody A block binding of PD-L1 to CD80. 178G7 competes with previously identified surrogate antibody 10F.9G2 known to block PD-L1/PD-interaction and is a known surrogate for anti-human antibodies in clinic (Eppihimer et al. Microcirculation 2002:9(2):133).

The HC of 178G7 is SEQ ID NO: 11, and the LC of 178G7 is SEQ ID NO: 12.

CT26 syngenic tumor cells are culture in media (RPMI1640; 10% FBS; 1 mM NA Pyruvate; 2 mM L-Glutamine). Cells are passaged twice a week before balb/C mice are seeded with 1×10⁶ CT26 cells. Tumors are allowed to grow for 6 days to between 50-100 mm³ in size before treatment. At 6 days of growth, are injected intra-peritoneal with either 60 mg/kg of Control IgG, 500 μg flat dose of 178G7, 60 mg/kg of CS7, or a combination of 500 μg flat dose of 178G7, together with 60 mg/kg of CS7. 178G7 is given once, every 7 days, with CS7 and Control IgG dosed two times per 7 days. Animal are dosed for 3 weeks and then followed for 85 days. If tumor burden becomes greater than 2500 mm³ animals are sacrificed due to progressive disease. Animals with tumors that have not reached 2500 mm³ after all animals in control groups reached their survival limit are considered partial responders if the tumor volume measured is smaller than two standard deviations from the mean of tumor volume of the control IgG animals, for at least 14 days. Animals which no longer have measurable tumor burden after 60 study days are considered complete responders. If an animal is alive after 85 days, and has rejected its tumor or a tumor has not reached 2500 mm³, it is re-challenged with CT26 tumor cells to test the generation of immunologic memory after therapy. After 105 days, any animal remaining in the study without measureable tumor burden, which had previously completely rejected their initial tumor challenge, is considered to have rejected secondary tumor challenge.

In experiments performed essentially as described, treatment with control IgG had no delay on tumor growth. All animals had reached or exceeded their tumor burden limit by 34 days in the study. At day 60 there were no complete regression without therapy. Therapy with CS7 alone produced a delay in tumor growth for 3/15 animals in the study. No complete regressions were observed with CS7 treatment alone. Anti-PD-L1 therapy with 178G7 caused tumor delay in 10 of 15 animals. Complete regressions were reached in 5 of these animals, with an overall complete response rate of 5/15 animals treated with 178G7 alone. Concurrent combination therapy with CS7 and 178G7 caused a delay of 8 of 10 animals treated. Of the animals which showed tumor growth delay with combination CS7 and 178G7 therapy, 6 achieved complete regression. The number of complete responders measure is shown in Table 2.

To determine if mice, with rejected primary tumors, had achieved immunologic memory, all mice which had achieved complete responses were re-challenged with CT26 tumor cells. All mice which demonstrated complete regression of primary tumors rejected re-challenge (Table 2).

TABLE 2

| | | | Secondary Tumor Challenge | |
|---|---|---|---|---|
| | Primary Tumor | | Mice rejecting | % rejecting |
| | Complete | % Complete | secondary | secondary |
| Group | Responders | Responders | challenge | challenge |
| CS7 alone | 0/10 | 0 | N/A | N/A |
| 178G7 alone | 5/15 | 33 | 5/5 | 100% |
| CS7 + | 6/10 | 60 | 6/6 | 100% |
| 178G7 | | | | |

### EXAMPLE 3

### Efficacy Study of CSF-1R Antibody in Combination with PD-L1 Antibody in MC38 Syngenic Tumor Model

To test the range of the efficacy of CSF-1R antibody in combination with PD-L1 antibody, monotherapy and combinations are tested in the MC38 syngenic tumor model, which is a model that is considered to be a macrophage infiltration model.

Since the MC38 model is a mouse model, the CSF-1R antibody and PD-L1 antibody utilized in the study must each bind their respective mouse target protein as Antibody 1 and Antibody A are not cross-reactive in mice, CS7 and 178G7, as described herein, are used as surrogates.

MC38 syngenic tumor cells are cultured in media (RPMI1640; 10% FBS; 1 mM NA Pyruvate; 2 mM L-Glutamine). Cells are passaged twice a week before C57BL/6 mice are seeded with 5×10⁵ MC38 cells. Tumors are allowed to grow for 6 days to between 25-75 mm³ in size before treatment. At 3 days of growth, cells are injected intra-peritoneal with either 60 mg/kg of Control IgG, 500 μg flat dose of anti-PD-L1 178G7, 60 mg/kg of CS7, or a combination of 500 μg flat dose of 178G7, together with 60 mg/kg of CS7. 178G7 is given once, every 7 days, with CS7 and Control IgG dosed two times per 7 days. Animal are dosed for 3 weeks and then followed for up to 85 days. If tumor burden becomes greater than 2500 mm³ animals are sacrificed due to progressive disease. Animals with tumors that have not reached 2500 mm³ after all animals in control groups reached their survival limit are considered partial responders if the tumor volume measured is smaller than two standard deviations from the mean of tumor volume of the control IgG animals, for at least 14 days.

In experiments performed essentially as described, treatment with control IgG had no delay on tumor growth. All animals had reached or exceeded their tumor burden limit by 20 days in the study. Therapy with CS7 alone did not produce a significant delay for animals in the study. No complete regressions were observed with CS7 treatment alone. Anti-PD-L1 therapy with 178G7 caused partial response in 4 of 10 animals. Concurrent combination therapy with IMC-CS7 and 178G7 caused a partial response, resulting in a delay in tumor growth in 7 of 10 animals treated. (Table 3)

TABLE 3

Primary Tumor

| | Partial | % Partial |
|---|---|---|
| Group | Responders | Responders |
| CS7 alone | 2/10 | 20 |
| 178G7 alone | 4/10 | 40 |
| CS7 + 178G7 | 7/10 | 70 |

### EXAMPLE 4

A Phase 1a/1b Study of Anti-PD-L1 Antibody, Antibody A, Administered Alone or in Combination with Anti-CSF-1R Antibody, Antibody 1, in Advanced Refractory Solid Tumors

### Study Design

This multicenter Phase 1 study is a Phase 1a dose escalation and Phase 1b dose expansion. Along with Antibody A targeting PD-L1, the combination partners in open-label dose escalation Phase 1a include Antibody 1 targeting CSF-1R. The Phase 1b expansion is to assess the safety, tolerability, and preliminary antitumor activity of Antibody A as monotherapy and in combination with Antibody 1 in study population determined based upon the results of Phase 1a.

The Recommended Phase 2 (RP2D) dose to be tested in the Phase 1b expansion will be identified in Phase 1a. The RP2D will be confirmed or may change based on the combined data from both Phases 1a and 1b.

### Study Objectives

The primary objective of this study is to assess the safety and tolerability of Antibody A for PD-L1, administered as monotherapy and in combination with Antibody 1 for CSF-1R to patients with advanced solid tumors.

The secondary objectives of this study are to assess the pharmacokinetics and preliminary antitumor activity of Antibody A administered as monotherapy and in combination with Antibody 1 in patients with advanced solid tumors.

TABLE 4

| Phase 1a- | Study Drug(s) | | | Treatment |
|---|---|---|---|---|
| Treatment | (route of | Dose | Dose | Cycle |
| Arm | administration) | Levels | Frequency | Duration |
| 1 | Antibody A | 70, 200, | Q2W | 28 Days |
| | (anti-PD-L1) (IV) | 700 mg | | |
| 2 | Antibody 1 | 50-100 mg | QW | 28 Days |
| | (anti-CSF-1R) (IV) | 70, 200, | Q2W | 28 Days |
| | Antibody A | 700 mg | | |
| | (anti-PD-L1) (IV) | | | |

Amino Acid Sequences

(HCVR Antibody 1)
SEQ ID NO: 1
QDQLVESGGGVVQPGRSLRLSCAASGFITSSYGMHWVRQAPGEGLEWVAV
IWYDGSNKYYADSVKGRFTISRDNSKNTLYLQMNSLRAEDTAVYYCARGD
YEVDYGMDVWGQGTTVTVAS
(LCVR Antibody 1)
SEQ ID NO: 2
AIQLTQSPSSLSASVGDRVTITCRASQGISNALAWYQQKPGKAPKLLIYD
ASSLESGVPSRFSGSGSGTDFTLTISSLQPEDFATYYCQQFNSYPWTFGQ
GTKVEIK
(HC Antibody 1)
SEQ ID NO: 3
QDQLVESGGGVVQPGRSLRLSCAASGFTFSSYGMHWVRQAPGEGLEWVAV
IWYDGSNKYYADSVKGRFTISRDNSKNTLYLQMNSLRAEDTAVYYCARGD
YEVDYGMDVWGQGTLVTVASASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFTPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYNKSLSLSPGK
(LC Antibody 1)
SEQ ID NO: 4
ATQLTQSPSSLSASVGDRVTITCRASQGISNALAWYQQKPGKAPKLLIYD
ASSLESGVPSRFSGSGSGTDFTLTISSLQPEDFATYYCQQFNSYPWTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
(HCVR Antibody A)
SEQ ID NO: 5
QVQLVQSGAEVKKPGSSVKVSCKASGGTFSSYAISWVRQAPGQGLEWMGG
IIPIFGTANYAQKFQGRVITTADKSTSTAYMELSSLRSEDTAVYYCARSP
DYSPYYYYGMDVWGQGTTVTVSS
(LCVR Antibody A)
SEQ ID NO: 6
QSVLTQPPSASGTPGQRVTISCSGSSSNIGSNTVNWYQQLPGTAPKLLIY
GNSNRPSGVPDRFSGSKSGTSASLAISGLQSEDEADYYCQSYDSSLSGSV
FGGGIKLTVLG
(HC Antibody A)
SEQ ID NO: 7
QVQLVQSGAEVKKPGSSVKVSCKASGGTFSSYAISWVRQAPGQGLEWMGG
IIPIFGTANYAQKFQGRVTITADKSTSTAYMELSSLRSEDTAVYYCARSP
DYSPYYYYGMDVWGQGTTVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKRVEPKSCDKTHTCPPCPAPEAEGAPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPSSIEKTISKAKGQPR
EPQVYMPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK
(LC Antibody A)
SEQ ID NO: 8
QSVLTQPPSASGTPGQRVTISCSGSSSNIGSNTVNWYQQLPGTAPKLLIY
GNSNRPSGVPDRFSGSKSGTSASLAISGLQSEDEADYYCQSYDSSLSGSV
FGGGIKLTVLGQPKAAPSVTLFPPSSEELQANKATLVCLISDFYPGAVTV
AWKADSSPVKAGVETTTPSKQSNNKYAASSYLSLTPEQWKSHRSYSCQVT
HEGSTVEKTVAPAECS
(HCVR CS7)
SEQ ID NO: 9
DRQMVESGGGLVRPGRSLKLSCAASGFTFSNYYMAWVRLAPSKGLEWVAS
ISPSGDNTYYPDSVQGRFTISRDNAETTLYLQVDSLRSEDTATYYCATRH
YGYSFFDYWGQGVLVTVSS
(LCVR CS7)
SEQ ID NO: 10
DTVLTQSPALAVSPGERVTISCRASESVSTLIHWYQQKPGQQPKLLIFLA
SHLESGVPARFSGSGSGTDFTFTIDPVEADDTATYYCQQSWNDPPTFGGG
TKLELK
(HC 178G7)
SEQ ID NO: 11
QVQLQQSGADLAKPGSSVKISCKASGYNFNSYYINWIKQTTGQGLEYIGY
INTVSGTTKYSEKFKGKATLTVDKSSSTAFMQLSSLTPDDSAVYYCARGT
IVLDDYWGQGVKVTVSSAETTAPSVYPLAPGTALKSNSMVTLGCLVKGYF
PEPVTVTWNSGALSSGVHTFPAVLQSGLYTLTSSVTVPSSTWPSQTVTCN
VAHPASSTKVDKKIVPRNCGGDCKPCICTGSEVSSVFIFPSKPKDVLTIT
LTPKVTCVVVDISQDDPEVHFSWFVDDVEVHTAQTRPPEEQFNSTFRSVS
ELPILHQDWLNGRTFRCKVTSAAFPSPIEKTISKPEGRTQVPHVYTMSPT
KEEMTQNEVSITCMVKGFYPPDIYVEWQMNGQPQENYKNTPPTMDTDGSY
FLYSKLNVKKEKWQQGNTFTCSVLHEGLHNHHTEKSLSHSPGK
(LC 178G7)
SEQ ID NO: 12
DIVMTQTPSSQAVSAGEKVTMSCKSSQSLLYNEKKKNYLAWYQQKPGQSP
KLLIYWASTRESGVPDRFLGSGSGTDFTLTINSVQAEDLANYYCQQSYDF
PRTFGGGTKLELKRADAAPTVSIFPPSTEQLATGGASVVCLMNNFYPRDI
SVKWKIDGTERRDGVLDSVTDQDSKDSTYSMSSTLSLTKADYESHNLYTC
EVVHKTSSSPVVKSFNRNEC
(HCDR1 Antibody 1)
SEQ ID NO: 13
SYGMH
(HCDR2 Antibody 1)
SEQ ID NO: 14
VIWYDGSNKYYADSVKG
(HCDR3 Antibody 1)
SEQ ID NO: 15
GDYEVDYGMDV
(LCDR1 Antibody 1)
SEQ ID NO: 16
RASQGISNALA
(LCDR2 Antibody 1)
SEQ ID NO: 17
DASSLES
(LCDR3 Antibody 1)
SEQ ID NO: 18
QQFNSYPWT
(HCDR1 Antibody A)
SEQ ID NO: 19
KASGGTFSSYAIS
(HCDR2 Antibody A)
SEQ ID NO: 20
GIIPIFGTANYAQKFQG
(HCDR3 Antibody A)
SEQ ID NO: 21
ARSPDYSPYYYYGMDV
(LCDR1 Antibody A)
SEQ ID NO: 22
SGSSSNIGSNTVN
(LCDR2 Antibody A)
SEQ ID NO: 23
YGNSNRPS
(LCDR3 Antibody A)
SEQ ID NO: 24
QSYDSSLSGSV

1. A method of treating cancer, comprising administering in simultaneous, separate, or sequential combination to a patient in need thereof, effective amounts of a first antibody and a second antibody, wherein:
a. the first antibody comprises a light chain (LC) and a heavy chain (HC), wherein the LC comprises a light chain variable region (LCVR) and the HC comprises a heavy chain variable region (HCVR), wherein the LCVR has the amino acid sequence given in SEQ ID NO: 2, and the HCVR has the amino acid sequence given in SEQ ID NO: 1; and
b. the second antibody comprises a LC and a HC, wherein the LC comprises a LCVR and the HC comprises a HCVR, wherein the LCVR has the amino acid sequence given in SEQ ID NO: 6, and the HCVR has the amino acid sequence given in SEQ ID NO: 5.

2. The method of claim 1, wherein the LC of the first antibody has the amino acid sequence given in SEQ ID NO: 4, and the HC of the first antibody has the amino acid sequence given in SEQ ID NO: 3.

3. The method of claim 2, wherein the LC of the second antibody has the amino acid sequence given in SEQ ID NO: 8, and the HC of the second antibody has the amino acid sequence given in SEQ ID NO: 7.

4. The method of claim 3, wherein:
a. the first antibody comprises two light chains and two heavy chains, wherein each light chain has the amino acid sequence given in SEQ ID NO: 4, and each heavy chain has the amino acid sequence given in SEQ ID NO: 3; and
b. the second antibody comprises two light chains and two heavy chains, wherein each light chain has the amino acid sequence given in SEQ ID NO: 8, and each heavy chain has the amino acid sequence given in SEQ ID NO: 7.

5. The method of claim 4, wherein the cancer is a solid tumor.

6. The method of claim 4, wherein the cancer has macrophage infiltration.

7. The method of claim 4, wherein the cancer is breast cancer, prostate cancer, lung cancer, head and neck cancer, colorectal cancer, pancreatic cancer, gastric cancer, kidney cancer, bladder cancer, melanoma, ovarian cancer, esophageal cancer, soft tissue sarcoma, or hepatocellular carcinoma.

8-35. (canceled)

36. A kit comprising a first antibody and a second antibody, wherein:
a. the first antibody comprises a light chain (LC) and a heavy chain (HC), wherein the LC comprises a light chain variable region (LCVR) and the HC comprises a heavy chain variable region (HCVR), wherein the LCVR has the amino acid sequence given in SEQ ID NO: 2, and the HCVR has the amino acid sequence given in SEQ ID NO: 1; and
b. the second antibody comprises a LC and a HC, wherein the LC comprises a LCVR and the HC comprises a HCVR, wherein the LCVR has the amino acid sequence given in SEQ ID NO: 6, and the HCVR has the amino acid sequence given in SEQ ID NO: 5.

37. The kit of claim 36, wherein the LC of the first antibody has the amino acid sequence given in SEQ ID NO: 4, and the HC of the first antibody has the amino acid sequence given in SEQ ID NO: 3.

38. The kit of claim 37, wherein the LC of the second antibody has the amino acid sequence given in SEQ ID NO: 8, and the HC of the second antibody has the amino acid sequence given in SEQ ID NO: 7.

39. The kit of claim 38, wherein:
a. the first antibody comprises two light chains and two heavy chains, wherein each light chain has the amino acid sequence given in SEQ ID NO: 4, and each heavy chain has the amino acid sequence given in SEQ ID NO: 3; and
b. the second antibody comprises two light chains and two heavy chains, wherein each light chain has the amino acid sequence given in SEQ ID NO: 8, and each heavy chain has the amino acid sequence given in SEQ ID NO: 7.
